SEQUENCE LISTING (18 sequences)

SEQ ID NO: 1 - DNA, 24 bp, Artificial
  synthetic oligonucleotide
tgcagccaaagtgaagagggaaga

SEQ ID NO: 2 - DNA, 24 bp, Artificial
  synthetic oligonucleotide
catagcgagcagcccaaagaagaa

SEQ ID NO: 3 - DNA, 20 bp, Artificial
  synthetic oligonucleotide
actgtcaacttcggggacac

SEQ ID NO: 4 - DNA, 20 bp, Artificial
  synthetic oligonucleotide
catgcctaaacgcttcatca

SEQ ID NO: 5 - DNA, 20 bp, Artificial
  synthetic oligonucleotide
tcatcgtctgctcctcctct

SEQ ID NO: 6 - DNA, 20 bp, Artificial
  synthetic oligonucleotide
aggtgtcatcagcagccttt

SEQ ID NO: 7 - DNA, 20 bp, Artificial
  synthetic oligonucleotide
caatcaggtggtggtgtcag

SEQ ID NO: 8 - DNA, 20 bp, Artificial
  synthetic oligonucleotide
gctcctggactgttttctgc

SEQ ID NO: 9 - DNA, 21 bp, Artificial
  synthetic oligonucleotide
aagtcgcctcgaagatacaca

SEQ ID NO: 10 - DNA, 21 bp, Artificial
  synthetic oligonucleotide
aaggagagaacactgctcgtg

SEQ ID NO: 11 - DNA, 20 bp, Artificial
  synthetic oligonucleotide
ggacagagacgacaagcaca

SEQ ID NO: 12 - DNA, 20 bp, Artificial
  synthetic oligonucleotide
catctgtgtagggcatgtgg

SEQ ID NO: 13 - DNA, 23 bp, Artificial
  synthetic oligonucleotide
tacaccactctggcagccacact

SEQ ID NO: 14 - DNA, 24 bp, Artificial
  synthetic oligonucleotide
cggtgggtacattggtgacagaac

SEQ ID NO: 15 - DNA, 20 bp, Artificial
  synthetic oligonucleotide
gcaccgtcaaggctgagaac

SEQ ID NO: 16 - DNA, 20 bp, Artificial
  synthetic oligonucleotide
atggtggtgaagacgccagt

SEQ ID NO: 17 - DNA, 25 bp, Artificial
  synthetic oligonucleotide
gtaaatgggattccaacacgaacaa

SEQ ID NO: 18 - DNA, 24 bp, Artificial
  synthetic oligonucleotide
tgtcgtgcagtaagaacccaactc

(19) United States
(12) Patent Application Publication
(10) Pub. No.: US 2021/0009947 A1
(43) Pub. Date: Jan. 14, 2021

(54) METHODS AND MATERIALS FOR MAINTAINING AND EXPANDING STEM CELL-DERIVED HEPATOCYTE-LIKE CELLS

(71) Applicant: BioE LLC, Bloomington, MN (US)

(72) Inventor: Daniel Patrick Collins, Lino Lakes, MN (US)

(21) Appl. No.: 16/926,299

(22) Filed: Jul. 10, 2020

Related U.S. Application Data
(60) Provisional application No. 62/873,668, filed on Jul. 12, 2019.

(51) Int. Cl. C12N 5/071; C12M 1/00

(52) U.S. Cl. CPC C12N 5/067; C12M 23/20; C12N 2501/39; C12N 2500/25; C12N 2501/11; C12N 2501/115; C12N 2500/30; C12N 2501/12; C12N 2501/125; C12N 2501/237; C12N 2501/155; C12N 2501/2301; C12N 2500/62; C12N 2501/119

(57) ABSTRACT

Culture medium formulations are described that are capable of large-scale expansion of stem cell-derived hepatocyte-like cells while sustaining the activity of those cells to maintain the phenotype and biological characteristics of normal hepatocytes.

### CROSS-REFERENCE TO RELATED APPLICATIONS

This application claims the benefit of U.S. Provisional Application Ser. No. 62/873,668, filed Jul. 12, 2019. The disclosure of the prior application is considered part of (and is incorporated by reference in) the disclosure of this application.

### TECHNICAL FIELD

This document relates to compositions that can be used as cell culture media formulations, and more particularly to cell culture media formulations that are supportive of the large-scale expansion of stem cell-derived differentiated hepatocyte-like cells while maintaining their hepatocyte-like biological characteristics.

### BACKGROUND

Livers and liver cells play a large role in life-saving transplantation procedures and in drug discovery and toxicology. While there is a large need for donor livers to transplant, the actual availability (<8,000/yr) leaves that large need unmet. Liver cells, isolated from post-mortum donations, are used in drug discovery and toxicology due to the liver's central role in drug metabolism. Liver cells obtained for this purpose comprise a >$1B industry supplying cells for drug developers and contract research organizations sub-contracting to larger drug developers. The inherent variability of the different lots of cells (liver cells are obtained from different donors) with regards to drug interactions complicate the interpretation of in vitro toxicology experiments. A different source for the creation of hepatocytes for both purposes (therapeutic and toxicologic) that is stable and reproducible could provide the platform needed for the development of them.

In the pursuit of the goal of a stable source of hepatocyte-like cells for drug discovery and toxicology studies, a number of different approaches have been taken with different sources of stem cells. The vast majority of commercial efforts have been utilizing either embryonic stem cells (ESC) or induced pluripotent stem cells (iPS). Other studies have suggested that cells sources including umbilical cord blood non-hematopoietic stem cells could provide a potential source of cells for the development of liver and pancreatic cells. Most of these efforts have resulted in cells that have significantly reduced function when compared to donor-derived primary hepatocytes.

Currently, drug discovery and toxicology studies are dependent upon a renewable source of donated or cadaveric hepatocytes. They are critical to the industry, but they suffer from several insurmountable features. One limitation is that they are from individuals with different genetics and drug metabolism characteristics and the resultant hepatocytes are a limited lot size that cannot be renewed. Hepatocytes have a limited life in vitro and do not divide and expand under current known culture conditions. Consistency, standardization, and economy are great drivers for the development of these cells from genetically consistent sources like stem cells.

### SUMMARY

This document is based, at least in part, on composition (e.g., a maintenance medium and/or an expansion medium) that can support the large-scale expansion of stem cell-derived hepatocyte-like cells while maintaining their hepatocyte-like biological activities and phenotype. While using such compositions, hepatocyte-like cells that are differentiated from stem cells derived from primate embryonic stem cells (ESC) and from human cord blood-derived multi-lineage progenitor cells (MLPC) can be proliferated for an extended period of time while maintaining their hepatocyte biological characteristics. The compositions described herein can be used to facilitate the commercial scale production of stem cell-derived hepatocyte-like cells.

In one aspect, this document features a composition that includes a culture medium and an article of manufacture that includes such a culture medium. The medium includes hydrocortisone, bovine serum albumin, insulin, transferrin, selenium, epithelial growth factor, basic fibroblast growth factor, fibroblast growth factor 4, hepatocyte growth factor, stem cell factor, oncostatin M, bone morphogenic protein 4 and interleukin 1 beta. The culture medium can be effective to induce differentiation of human fetal blood multi-lineage progenitor cells (MLPC) or a clonal line of human fetal blood MLPC to cells having a hepatocyte phenotype. The composition can be effective in the long-term growth and maintenance of primate embryonic stem cell-derived hepatocyte-like cells. The composition further can include an antibiotic such as penicillin, streptomycin or gentamycin. The composition further can include dimethylsulfoxide (DMSO) and/or retinoic acid. The composition further can include at least one antibody, the at least one antibody having binding affinity for alkaline phosphatase, alpha fetoprotein, albumin, c-reactive protein, hepatocyte growth factor receptor, complement factor VII, complement factor IX, nestin, SOX-17, P450 CYP 1A2, P450 CYP 3A4, asialo-glycoprotein receptor 1, hepatocyte nuclear factor-4, GATA-4, or alpha-1-antitrypsin (e.g., two or more antibodies, three or more antibodies, four or more antibodies, five or more antibodies, six or more antibodies, seven or more antibodies, eight or more antibodies, nine or more antibodies, ten or more antibodies, eleven or more antibodies, twelve or more antibodies, thirteen or more antibodies, fourteen or more antibodies, or fifteen antibodies). In some embodiments, albumin production and/or urea production can be measured.

In some embodiments, this document features an article of manufacture that includes a composition described herein, wherein the composition is housed in a container. The container can be a vial, bottle, or a bag. Such an article of manufacture can include a population of cells having a hepatocyte phenotype (e.g., a clonal population of cells having a hepatocyte phenotype).

In another aspect, this document features a method of producing a population of cells having a hepatocyte phenotype. The method includes a) providing a collagen-coated culturing device housing a purified population of MLPC or a clonal line of MLPC (e.g., an immortalized MLPC comprising a nucleic acid encoding a telomerase reverse transcriptase); culturing the purified population of MLPC or the clonal line of MLPC with a differentiation medium containing Activin A, until cells having an endodermal precursor phenotype are obtained, further culturing the cells having the endodermal precursor phenotype in a differentiation medium comprising hydrocortisone, bovine serum albumin, insulin, transferrin, selenium, epithelial growth factor, basic fibroblast growth factor, fibroblast growth factor 4, hepatocyte growth factor, stem cell factor, oncostatin M, bone morphogenic protein 4 and interleukin 1 beta, to obtain cells having the committed hepatocyte precursor cell phenotype, and further culturing the cells in the differentiation medium in the presence of DMSO and retinoic acid to obtain cells having the hepatocyte phenotype. The method further can include testing the cells having the hepatocyte phenotype for one or more (e.g., two, three, four, five, six, seven, eight, nine, ten, eleven, twelve, thirteen, fourteen, or all) of alkaline phosphatase, alpha fetoprotein, albumin, c-reactive protein, hepatocyte growth factor receptor, complement factor VII, complement factor IX, nestin, SOX-17, P450 CYP 1A2, P450 CYP 3A4, asialo-glycoprotein receptor 1, hepatocyte nuclear factor-4, GATA-4, and alpha-1-antitrypsin. In some embodiments, albumin production and/or urea production can be measured.

This document also features a method of expanding a population of primate embryonic stem cell-derived hepatocyte-like cells. The method includes growing the cells in a differentiation medium comprising hydrocortisone, bovine serum albumin, insulin, transferrin, selenium, epithelial growth factor, basic fibroblast growth factor, fibroblast growth factor 4, hepatocyte growth factor, stem cell factor, oncostatin M, bone morphogenic protein 4 and interleukin 1 beta. The method can further include testing the cells having the hepatocyte phenotype for one or more (e.g., two, three, four, five, six, seven, eight, nine, ten, eleven, twelve, thirteen, fourteen, or all) of alkaline phosphatase, alpha fetoprotein, albumin, c-reactive protein, hepatocyte growth factor receptor, complement factor VII, complement factor IX, nestin, SOX-17, P450 CYP 1A2, P450 CYP 3A4, asialo-glycoprotein receptor 1, hepatocyte nuclear factor-4, GATA-4, and alpha-1-antitrypsin.

This document also features a kit that includes any of the compositions described herein, wherein the composition is housed within a container and a clonal population of cells having the hepatocyte phenotype. For example, a hepatocyte phenotype can include expression of all of alkaline phosphatase, alpha fetoprotein, albumin, c-reactive protein, hepatocyte growth factor receptor, complement factor VII, complement factor IX, nestin, SOX-17, P450 CYP 1A2, P450 CYP 3A4, asialo-glycoprotein receptor 1, hepatocyte nuclear factor-4, GATA-4, and alpha-1-antitrypsin. The clonal population of cells can be cryopreserved (e.g., with fetal bovine serum, human serum, or human serum albumin in combination with one or more of the following: DMSO, trehalose, and dextran).

In some embodiments, this document features a composition that includes a purified population of human fetal blood MLPC or a clonal line of human fetal blood MLPC and a medium effective to induce differentiation of the MLPC into cells having a hepatocyte phenotype, wherein the MLPC, prior to differentiation, are positive for CD9, CD105, CD106, CD90, negative for CD45, negative for CD34, and negative for SSEA-4. The differentiation medium can include hydrocortisone, transferrin, insulin, selenium, epidermal growth factor, hepatocyte growth factor, stem cell factor, basic fibroblast growth factor, fibroblast growth factor-4, oncostatin M, bone morphogenic protein 4, and Interleukin 1 beta. In some embodiments, a medium also can include Activin A (e.g., until cells having an endodermal precursor phenotype are obtained). In some embodiments, a medium also can include dimethyl sulfoxide and retinoic acid (e.g., until cells have a mature hepatocyte phenotype). The differentiation medium further can include an antibiotic, such as penicillin, streptomycin or gentamycin.

In another aspect, this embodiment features a method for producing a population of cells having a hepatocyte phenotype from human fetal blood. The method includes contacting a human fetal blood sample with a composition, the composition including dextran, anti-CD15 antibody, and Ca′ and Mg′ ions; allowing the sample to partition into an agglutinate and a supernatant phase; recovering cells from the supernatant phase; purifying MLPC from the recovered cells by adherence to a solid substrate, wherein the MLPC are positive for CD9, CD105, CD106, CD90, negative for CD45, negative for CD34, and negative for SSEA-4. In some embodiments, the method further can include producing a clonal line of MLPC from the MLPC having the fibroblast morphology before culturing with the differentiation medium. Such compositions further can include a cryopreservative (e.g., DMSO such as 1 to 10% DMSO). The cryopreservative can be fetal bovine serum, human serum, or human serum albumin in combination with one or more of the following: DMSO, trehalose, and dextran. For example, the cryopreservative can be human serum, DMSO, and trehalose, or fetal bovine serum and DMSO.

In yet another aspect, this embodiment features a clonal population of cells having a hepatocyte phenotype and compositions containing such clonal populations. In one embodiment, a composition includes a clonal population of cells having a hepatocyte phenotype and a culture medium. Such compositions further can include a cryopreservative (e.g., DMSO such as 1 to 10% DMSO). The cryopreservative can be fetal bovine serum, human serum, or human serum albumin in combination with one or more of the following: DMSO, trehalose, and dextran. For example, the cryopreservative can be human serum, DMSO, and trehalose, or fetal bovine serum and DMSO.

In another aspect, this document provides a method for differentiating primate embryonic stem cells (e.g., marmoset embryonic stem cells) to a hepatocyte-like phenotype using a medium that can include Activin A, fibroblast growth factor 2, bone morphogenic protein 4, hepatocyte growth factor, and oncostatin M. Such compositions further can include a cryopreservative (e.g., DMSO such as 1 to 10% DMSO). The cryopreservative can be fetal bovine serum, human serum, or human serum albumin in combination with one or more of the following: DMSO, trehalose, and dextran. For example, the cryopreservative can be human serum, DMSO, and trehalose, or fetal bovine serum and DMSO.

This document also features a method of utilization for the manufacture of stem cell-derived hepatocyte-like cells for commercial level cell production.

This document also features a kit containing a basal medium (e.g., Williams E medium), growth factors (e.g., hydrocortisone, bovine serum albumin, insulin, transferrin, selenium, epithelial growth factor, basic fibroblast growth factor, fibroblast growth factor 4, hepatocyte growth factor, stem cell factor, oncostatin M, bone morphogenic protein 4 and interleukin 1 beta), and one or more antibiotics that can be combined to create a final medium, as well as, instructions for use that would enable users of this medium to successfully expand their stem cell-derived hepatocyte-like cells.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a schematic of the procedural steps to isolate MLPC from human umbilical cord blood.

FIG. 2 is a schematic of the procedural steps to differentiate MLPC to hepatocyte-like cells.

FIG. 3 is a schematic of the procedural steps to differentiate primate ESC to hepatocyte-like cells.

FIG. 4 contains photomicrographs of MLPC- and primate ESC-derived hepatocyte-like cells.

FIGS. 5A and 5B are photomicrographs of PCR products resolved on a 1% agarose gel and visualized under UV light.

### DETAILED DESCRIPTION

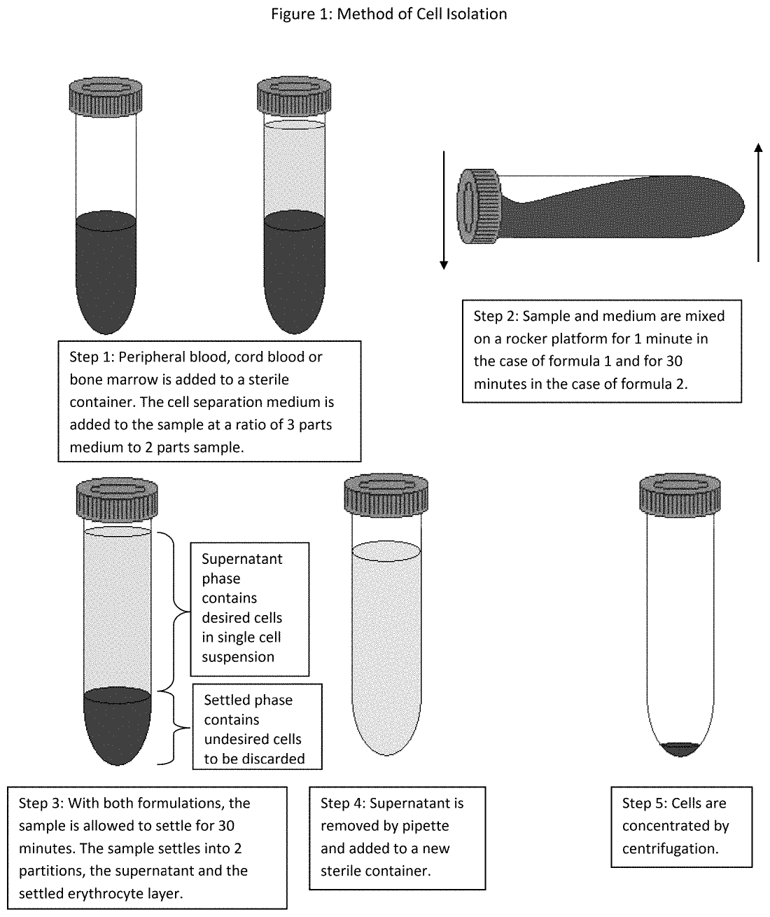

In general, this document provides methods and materials for maintaining and expanding stem cell-derived hepatocyte-like cells. Cells with a hepatocyte phenotype can be obtained by inducing differentiation, for example, of MLPC from human fetal blood. As described in U.S. Pat. Nos. 7,670,596, 7,622,108, and 7,875,54, fetal blood MLPC are distinguished from bone marrow-derived MSC, HSC, and USSC on the basis of their immunophenotypic characteristics, gene expression profile, morphology, and distinct growth pattern. MLPC can be isolated from fetal blood (e.g., cord blood) using the negative selection process and cell separation compositions disclosed in U.S. Pat. Nos. 7,160,723 and 7,476,547. FIG. 1 provides a schematic of the steps to isolate MLPC from human umbilical cord blood.

The liver and pancreas develop from an embryonic endodermal tissue in the mid-thoracic area. The tissue that differentiates into the pancreas is most influenced by their proximity to the developing gastro-intestinal system. The tissue that develops into the liver is influenced by its proximity to the developing cardiac tissue. The systems that were developed to direct differentiation to these different tissue types were based on the development in the embryo. First develop the cells into the common endodermal tissue of the two organs then add growth factors that would most closely replicate the conditions that cause the cells to divert their development pathway to either more fully developed liver or pancreatic tissues.

As described herein, progression of MLPC and primate ESC to the final hepatocyte-like state was confirmed using fluorescent microscopy to assess protein and cell-specific marker expression and various functionality tests to determine the completeness of the differentiation process. For example, a hepatocyte phenotype can be confirmed by the presence of one or more of alkaline phosphatase, alpha fetoprotein, albumin, c-reactive protein, hepatocyte growth factor receptor, complement factor VII, complement factor IX, nestin, SOX-17, P450 CYP 1A2, P450 CYP 3A4, asialo-glycoprotein receptor 1, hepatocyte nuclear factor-4, GATA-4, and alpha-1-antitrypsin. For example, a hepatocyte phenotype can be confirmed by the presence of two, three, four, five, six, seven, eight, nine, ten, eleven, twelve, thirteen, fourteen, or all of alkaline phosphatase, alpha fetoprotein, albumin, c-reactive protein, hepatocyte growth factor receptor, complement factor VII, complement factor IX, nestin, SOX-17, P450 CYP 1A2, P450 CYP 3A4, asialo-glycoprotein receptor 1, hepatocyte nuclear factor-4, GATA-4, and alpha-1-antitrypsin. In addition, albumin production, urea production, clotting factors, and activity of P450 CYP activity (essential for use in drug toxicity applications) can be measured. Albumin production has been measured from the hepatocyte cells differentiated as described herein.

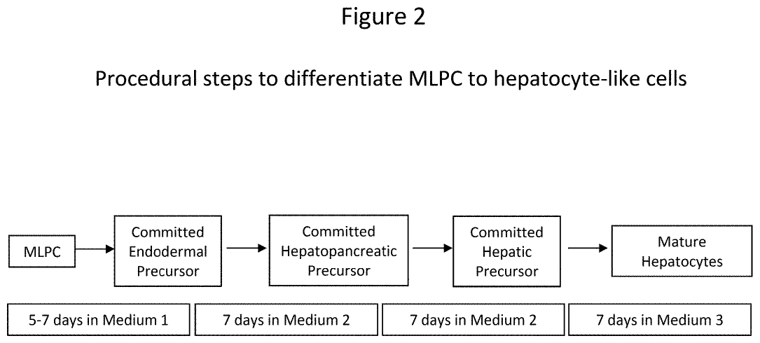

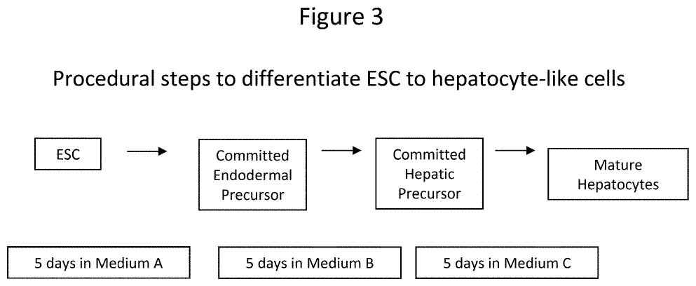

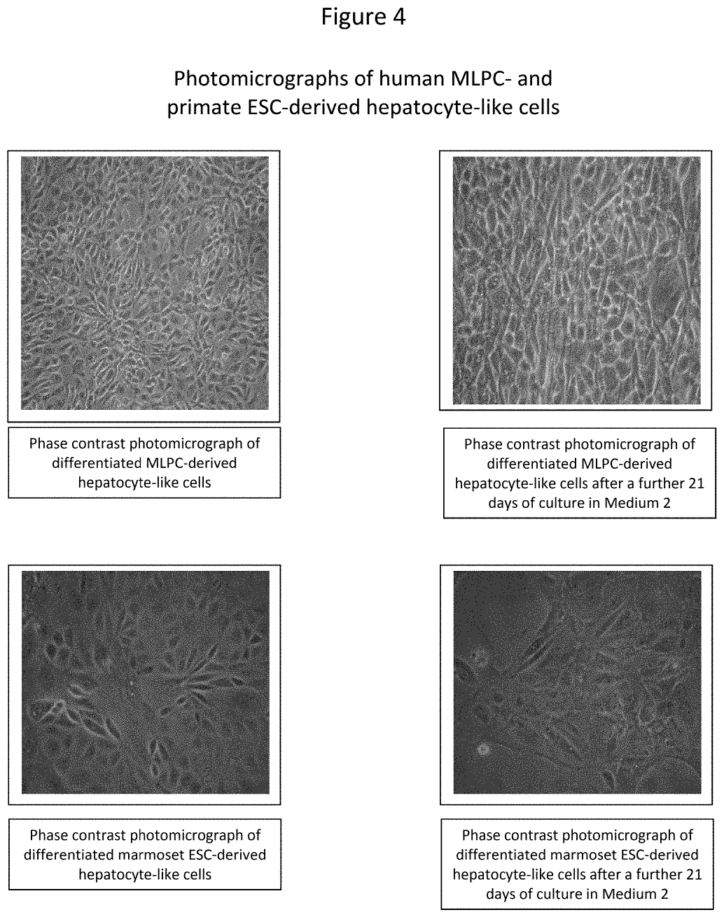

The differentiation of MLPC to hepatocyte-like cells can be accomplished in several phases, using different culture conditions. A diagram of the procedural steps to differentiate MLPC to hepatocyte-like cells is shown in FIG. 2. The first stage is to push the cells to a committed endodermal fate. As a fertilized ovum grows to be a hollow ball of cells called the blastocyte, the cells divide into three distinct layers called (from outside to in) the ectoderm, the mesoderm, and the endoderm. The ectoderm is responsible for the development of the skin, brain and neural tissue. The mesoderm is responsible for the development of the bone, muscle, connective and blood tissues. The endoderm is responsible for the development of pancreatic, liver, lung, and endocrine tissues.

Efforts to create functional hepatocytes from cord blood-derived MLPC have achieved many of the goals consistent with functional hepatocytes, including production of albumin and urea, expression of surface membrane asialo-gylcoprotein receptors and translocation of hepatocyte nuclear factor-4 from the cytoplasm to the nucleus. Parallel studies with primate embryonic stem cell derived hepatocyte-like cells have demonstrated similar results.

Among the important discoveries achieved by these studies was the formulation of a medium that supports the growth and expansion of differentiated stem cell-derived hepatocyte-like cells. This medium supports the expansion of these differentiated cells while maintaining the hepatocyte morphology, protein expression and preventing the reversion of the differentiated cells to more primitive states. The medium includes hydrocortisone, bovine serum albumin, insulin, transferrin, selenium, epithelial growth factor, basic fibroblast growth factor, fibroblast growth factor 4, hepatocyte growth factor, stem cell factor, oncostatin M, bone morphogenic protein 4 and interleukin 1 beta. For example, the medium can include about 5 mM hydrocortisone (e.g., hydrocortisone-21-hemisuccinate, about 80 ng/ml epithelial growth factor, about 20 ng/ml fibroblast growth factor basic, about 20 ng/ml fibroblast growth factor 4, about 40 ng/ml hepatocyte growth factor, about 40 ng/ml stem cell factor, about 20 ng/ml Oncostatin M, about 20 ng/ml bone morphogenic protein 4, and about 10 ng/ml Interleukin 1 beta. In some embodiments, the medium also can include an antibiotic and/or DMSO and/or retinoic acid.

### Cell Separation Compositions

MLPC can be isolated from fetal blood (e.g., cord blood) using the negative selection process and cell separation compositions disclosed in U.S. Pat. Nos. 7,160,723 and 7,476,547. Such cell compositions can include dextran and one or more antibodies against (i.e., that have binding affinity for) a cell surface antigen. Dextran is a polysaccharide consisting of glucose units linked predominantly in alpha (1 to 6) mode. Dextran can cause stacking of erythrocytes (i.e., rouleau formation) and thereby facilitate the removal of erythroid cells from solution. Antibodies against cell surface antigens can facilitate the removal of blood cells from solution via homotypic agglutination (i.e., agglutination of cells of the same cell type) and/or heterotypic agglutination (i.e., agglutination of cells of different cell types). For example, a cell separation composition can include dextran and antibodies against glycophorin A, CD15, and CD9. Cell separation compositions also can contain antibodies against other blood cell surface antigens including, for example, CD2, CD3, CD4, CD8, CD72, CD16, CD41a, HLA Class I, HLA-DR, CD29, CD11a, CD11b, CD11c, CD19, CD20, CD23, CD39, CD40, CD43, CD44, CDw49d, CD53, CD54, CD62L, CD63, CD66, CD67, CD81, CD82, CD99, CD100, Leu-13, TPA-1, surface Ig, and combinations thereof. Thus, cell separation compositions can be formulated to selectively agglutinate particular types of blood cells.

Typically, the concentration of anti-glycophorin A antibodies in a cell separation composition ranges from 0.1 to 15 mg/L (e.g., 0.1 to 10 mg/L, 1 to 5 mg/L, or 1 mg/L). Anti-glycophorin A antibodies can facilitate the removal of red cells from solution by at least two mechanisms. First, anti-glycophorin A antibodies can cause homotypic agglutination of erythrocytes since glycophorin A is the major surface glycoprotein on erythrocytes. In addition, anti-glycophorin A antibodies also can stabilize dextran-mediated rouleau formation. Exemplary monoclonal anti-glycophorin A antibodies include, without limitation, 107FMN (Murine IgG1 isotype), YTH89.1 (Rat IgG2b isotype), 2.2.2.E7 (Murine IgM isotype; BioE, St. Paul, Minn.), and E4 (Murine IgM isotype). See e.g., M. Vanderlaan et al., Molecular Immunology 20:1353 (1983); Telen M. J. and Bolk, T. A., Transfusion 27: 309 (1987); and Outram S. et al., Leukocyte Research. 12:651 (1988).

The concentration of anti-CD15 antibodies in a cell separation composition can range from 0.1 to 15 mg/L (e.g., 0.1 to 10, 1 to 5, or 1 mg/L). Anti-CD15 antibodies can cause homotypic agglutination of granulocytes by crosslinking CD15 molecules that are present on the surface of granulocytes. Anti-CD15 antibodies also can cause homotypic and heterotypic agglutination of granulocytes with monocytes, NK-cells and B-cells by stimulating expression of adhesion molecules (e.g., L-selectin and beta-2 integrin) on the surface of granulocytes that interact with adhesion molecules on monocytes, NK-cells and B-cells. Heterotypic agglutination of these cell types can facilitate the removal of these cells from solution along with red cell components. Exemplary monoclonal anti-CD15 antibodies include, without limitation, AHN1.1 (Murine IgM isotype), FMC-10 (Murine IgM isotype), BU-28 (Murine IgM isotype), MEM-157 (Murine IgM isotype), MEM-158 (Murine IgM isotype), 324.3.B9 (Murine IgM isotype; BioE, St. Paul, Minn.), and MEM-167 (Murine IgM isotype). See e.g., Leukocyte typing IV (1989); Leukocyte typing II (1984); Leukocyte typing VI (1995); Solter D. et al., Proc. Natl. Acad. Sci. USA 75:5565 (1978); Kannagi R. et al., J. Biol. Chem. 257:14865 (1982); Magnani, J. L. et al., Arch. Biochem. Biophys 233:501 (1984); Eggens I. et al., J. Biol. Chem. 264:9476 (1989).

The concentration of anti-CD9 antibodies in a cell separation composition can range from 0.1 to 15, 0.1 to 10, 1 to 5, or 1 mg/L. Anti-CD9 antibodies can cause homotypic agglutination of platelets. Anti-CD9 antibodies also can cause heterotypic agglutination of granulocytes and monocytes via platelets that have adhered to the surface of granulocytes and monocytes. CD9 antibodies can promote the expression of platelet p-selectin (CD62P), CD41/61, CD31, and CD36, which facilitates the binding of platelets to leukocyte cell surfaces. Thus, anti-CD9 antibodies can promote multiple cell-cell linkages and thereby facilitate agglutination and removal from solution. Exemplary monoclonal anti-CD9 antibodies include, without limitation, MEM-61 (Murine IgG1 isotype), MEM-62 (Murine IgG1 isotype), MEM-192 (Murine IgM isotype), FMC-8 (Murine IgG2a isotype), SN4 (Murine IgG1 isotype), 8.10.E7 (Murine IgM isotype; BioE, St. Paul, Minn.), and BU-16 (Murine IgG2a isotype). See e.g., Leukocyte typing VI (1995); Leukocyte typing II (1984); Von dem Bourne A. E. G. Kr. and Moderman P. N. (1989) In Leukocyte typing IV (ed. W. Knapp, et al), pp. 989-92, Oxford University Press, Oxford; Jennings, L. K., et al. In Leukocyte typing V, ed. S. F. Schlossmann et al., pp. 1249-51, Oxford University Press, Oxford (1995); Lanza F. et al., J. Biol. Chem. 266:10638 (1991); Wright et al., Immunology Today 15:588 (1994); Rubinstein E. et al., Seminars in Thrombosis and Hemostasis 21:10 (1995).

In some embodiments, a cell separation composition contains antibodies against CD41, which can selectively agglutinate platelets. In some embodiments, a cell separation composition contains antibodies against CD3, which can selectively agglutinate T-cells. In some embodiments, a cell separation composition contains antibodies against CD2, which can selectively agglutinate T-cells and NK cells. In some embodiments, a cell separation composition contains antibodies against CD72, which can selectively agglutinate B-cells. In some embodiments, a cell separation composition contains antibodies against CD16, which can selectively agglutinate NK cells and neutrophilic granulocytes. The concentration of each of these antibodies can range from 0.01 to 15 mg/L. Exemplary anti-CD41 antibodies include, without limitation, PLT-1 (Murine IgM isotype), CN19 (Murine IgG₁ isotype), and 8.7.C3 (Murine IgG₁ isotype). Non-limiting examples of anti-CD3 antibodies include OKT3 (Murine HIT3a (Murine IgG₂a isotype), SK7 (Murine IgG₁) and BC3 (Murine IgG₂a). Non-limiting examples of anti-CD2 antibodies include 7A9 (Murine IgM isotype), T11 (Murine IgG₁ isotype), and Leu5b (Murine IgG₂a Isotype). Non-limiting examples of anti-CD72 antibodies include BU-40 (Murine IgG₁ isotype) and BU-41 (Murine IgG₁ isotype). Non-limiting examples of anti-CD16 antibodies include 3G8 (Murine IgG).

As mentioned above, cell separation compositions can be formulated to selectively agglutinate particular blood cells. As an example, a cell separation composition containing antibodies against glycophorin A, CD15, and CD9 can facilitate the agglutination of erythrocytes, granulocytes, NK cells, B cells, and platelets. T cells, NK cells, and rare precursor cells such as MLPC, then can be recovered from solution. If the formulation also contained an antibody against CD3, T cells also could be agglutinated, and NK cells and rare precursors such as MLPC could be recovered from solution.

Cell separation compositions can contain antibodies against surface antigens of other types of cells (e.g., cell surface proteins of tumor cells). Those of skill in the art can use routine methods to prepare antibodies against cell surface antigens of blood, and other, cells from humans and other mammals, including, for example, non-human primates, rodents (e.g., mice, rats, hamsters, rabbits and guinea pigs), swine, bovines, and equines.

Typically, antibodies used in the composition are monoclonal antibodies, which are homogeneous populations of antibodies to a particular epitope contained within an antigen. Suitable monoclonal antibodies are commercially available, or can be prepared using standard hybridoma technology. In particular, monoclonal antibodies can be obtained by techniques that provide for the production of antibody molecules by continuous cell lines in culture, including the technique described by Kohler, G. et al., Nature, 1975, 256:495, the human B-cell hybridoma technique (Kosbor et al., Immunology Today 4:72 (1983); Cole et al., Proc. Natl. Acad. Sci. USA 80:2026 (1983)), and the EBV-hybridoma technique (Cole et al., “Monoclonal Antibodies and Cancer Therapy,” Alan R. Liss, Inc., pp. 77-96 (1983)).

Antibodies can be of any immunoglobulin class including IgG, IgM, IgE, IgA, IgD, and any subclass thereof. Antibodies of the IgG and IgM isotypes are particularly useful in cell separation compositions of the invention. Pentameric IgM antibodies contain more antigen binding sites than IgG antibodies and can, in some cases (e.g., anti-glycophorin A and anti-CD15), be particularly useful for cell separation reagents. In other cases (e.g., anti-CD9 antibodies), antibodies of the IgG isotype are particularly useful for stimulating homotypic and/or heterotypic agglutination.

Antibodies against cell surface antigens can be provided in liquid phase (i.e., soluble). Liquid phase antibodies typically are provided in a cell separation composition at a concentration between about 0.1 and about 15 mg/l (e.g., between 0.25 to 10, 0.25 to 1, 0.5 to 2, 1 to 2, 4 to 8, 5 to 10 mg/1).

Antibodies against cell surface antigens also can be provided in association with a solid phase (i.e., substrate-bound). Antibodies against different cell surface antigens can be covalently linked to a solid phase to promote crosslinking of cell surface molecules and activation of cell surface adhesion molecules. The use of substrate-bound antibodies can facilitate cell separation (e.g., by virtue of the mass that the particles contribute to agglutinated cells, or by virtue of properties useful for purification).

In some embodiments, the solid phase with which a substrate-bound antibody is associated is particulate. In some embodiments, an antibody is bound to a latex microparticle (0.5 to 10 microns in diameter) such as a paramagnetic bead (e.g., via biotin-avidin linkage, covalent linkage to COO groups on polystyrene beads, or covalent linkage to NH₂ groups on modified beads). In some embodiments, an antibody is bound to an acid-etched glass particle (e.g., via biotin-avidin linkage). In some embodiments, an antibody is bound to an aggregated polypeptide such as aggregated bovine serum albumin (e.g., via biotin-avidin linkage, or covalent linkage to polypeptide COO groups or NH₂ groups). In some embodiments, an antibody is covalently linked to a polysaccharide such as high molecular weight (e.g., >1,000,000 Mᵣ) dextran sulfate. In some embodiments, biotinylated antibodies are linked to avidin particles, creating tetrameric complexes having four antibody molecules per avidin molecule. In some embodiments, antibodies are bound to biotinylated agarose gel particles (One Cell Systems, Cambridge, Mass., U.S.A.) via biotin-avidin-biotinylated antibody linkages. Such particles typically are about 300-500 microns in size, and can be created in a sonicating water bath or in a rapidly mixed water bath.

Cell-substrate particles (i.e., particles including cells and substrate-bound antibodies) can sediment from solution as an agglutinate. Cell-substrate particles also can be removed from solution by, for example, an applied magnetic field, as when the particle is a paramagnetic bead. Substrate-bound antibodies typically are provided in a cell separation composition at a concentration between about 0.1 and about 50.0×10⁹ particles/1 (e.g., between 0.25 to 10.0×10⁹, 1 to 20.0×10⁹, 2 to 10.0×10⁹, 0.5 to 2×10⁹, 2 to 5×10⁹, 5 to 10×10⁹, and 10 to 30×10⁹ particles/1), where particles refers to solid phase particles having antibodies bound thereto by previously described methods.

Cell separation compositions also can contain divalent cations (e.g., Ca′ and Mg′). Divalent cations can be provided, for example, by a balanced salt solution (e.g., Hank's balanced salt solution). Ca′ ions reportedly are important for selectin-mediated and integrin-mediated cell-cell adherence.

Cell separation compositions also can contain an anticoagulant such as heparin. Heparin can prevent clotting and non-specific cell loss associated with clotting in a high calcium environment. Heparin also promotes platelet clumping. Clumped platelets can adhere to granulocytes and monocytes and thereby enhance heterotypic agglutination more so than single platelets. Heparin can be supplied as a heparin salt (e.g., sodium heparin, lithium heparin, or potassium heparin).

### Populations and Clonal Lines of MLPC

MLPC can be purified from human fetal blood using a cell separation composition described above. As used herein, “purified” means that at least 90% (e.g., 91, 92, 93, 94, 95, 96, 97, 98, or 99%) of the cells within the population are MLPC. As used herein, “MLPC” refers to fetal blood cells that are positive for CD9 and typically display a constellation of other markers such as CD13, CD73, and CD105. “MLPC population” refers to the primary culture obtained from the human fetal blood and uncloned progeny thereof. “Clonal line” refers to a cell line derived from a single cell. As used herein, a “cell line” is a population of cells able to renew themselves for extended periods of times in vitro under appropriate culture conditions. The term “line,” however, does not indicate that the cells can be propagated indefinitely. Rather, clonal lines described herein typically can undergo 75 to 100 doublings before senescing and without losing the properties of hepatocyte cells for this duration of propagation. Typically, an MLPC population is obtained by contacting a fetal blood sample with a cell separation composition described above and allowing the sample to partition into an agglutinate and a supernatant phase. For example, the sample can be allowed to settle by gravity or by centrifugation. Preferably, MLPC are purified from an umbilical cord blood sample that is less than 48 hours old (e.g., less than 24, 12, 8, or 4 hours post-partum). After agglutination, unagglutinated cells can be recovered from the supernatant phase. For example, cells in the supernatant phase can be recovered by centrifugation then washed with a saline solution and plated on a solid substrate (e.g., a plastic culture device such as a chambered slide or culture flask), using a standard growth medium with 10% serum (e.g., DMEM with 10% serum; RPMI-1640 with 10% serum, or mesenchymal stem cell growth medium with 10% serum (catalog #PT-3001, Cambrex, Walkersville, Md.). MLPC attach to the surface of the solid substrate while other cells, including T cells, NK cells and CD34⁺ HSC, do not and can be removed with washing. The MLPC change from the leukocyte morphology to the fibroblastic morphology between 3 days and 2 weeks post initiation of culture after which the cells enter logarithmic growth phase and will continue growing logarithmically as long as cultures are maintained at cell concentrations of less than about 1.5×10⁵ cells/cm².

Clonal lines can be established by harvesting the MLPC then diluting and re-plating the cells on a multi-well culture plate such that a single cell can be found in a well. Cells can be transferred to a larger culture flask after a concentration of 1 to 5×10⁵ cells/75 cm² is reached. Cells can be maintained at a concentration between 1×10⁵ and 5×10⁵ cells/75 cm² for logarithmic growth. See, e.g., U.S. Pat. Nos. 7,670,596 and 7,632,108

MLPC and cells having a hepatocyte phenotype can be assessed for viability, proliferation potential, and longevity using techniques known in the art. For example, viability can be assessed using trypan blue exclusion assays, fluorescein diacetate uptake assays, or propidium iodide uptake assays. Proliferation can be assessed using thymidine uptake assays or MTT cell proliferation assays. Longevity can be assessed by determining the maximum number of population doublings of an extended culture.

MLPC or cells having a hepatocyte phenotype can be immunophenotypically characterized using known techniques. Cells can be incubated with an antibody having binding affinity for a cell surface antigen such as hepatocyte growth factor receptor or asialo-glycoprotein receptor 1, or any other cell surface antigen. In some embodiments, cells can be fixed and permeabilized and incubated with an antibody that binds to an antigen internal to the cell. The antibody can be detectably labeled (e.g., fluorescently or enzymatically) or can be detected using a secondary antibody that is detectably labeled. Alternatively, the cell surface antigens on MLPC or cells having a hepatocyte phenotype can be characterized using flow cytometry and fluorescently labeled antibodies.

As described herein, the cell surface antigens present on MLPC can vary, depending on the stage of culture. Early in culture when MLPC display a leukocyte-like morphology, MLPC are positive for CD9 and CD45, SSEA-4 (stage-specific embryonic antigen-4), CD34, as well as CD13, CD29, CD44, CD73, CD90, CD105, stem cell factor, STRO-1 (a cell surface antigen expressed by bone marrow stromal cells), SSEA-3 (galactosylgloboside), and CD133, and are negative for CD15, CD38, glycophorin A (CD235a), and lineage markers CD2, CD3, CD4, CD5, CD7, CD8, CD10, CD11b, CD16, CD19, CD20, CD21, CD22, CD33, CD36, CD41, CD61, CD62E, CD72, HLA-DR, and CD102. After transition to the fibroblastic morphology, MLPC remain positive for CD9, CD13, CD29, CD73, CD90, and CD105, are positive for CD106, and become negative for CD34, CD41, CD45, stem cell factor, STRO-1, SSEA-3, SSEA-4, and CD133. At all times during in vitro culture, the undifferentiated MLPC are negative for CD15, CD38, glycophorin A (CD235a), and lineage markers CD2, CD3, CD4, CD5, CD7, CD8, CD10, CD11b, CD16, CD19, CD20, CD21, CD22, CD33, CD36, CD41, CD61, CD62E, CD72, HLA-DR, and CD102.

Bone marrow-derived MSC and MAPC as well as the cord blood-derived USSC have been described as being derived from a CD45⁻/CD34⁻ cell population. MLPC are distinguished from those cell types as being a CD45⁺/CD34⁺ derived cell. Additionally, the presence and persistence of CD9 on the fetal blood-derived MLPC at all stages of maturation further distinguishes MLPC from MSC and MAPC, which do not possess CD9 as a marker. CD9 is expressed as a marker on human embryonic stem cells. MLPC, which share the hematopoietic markers CD45, CD133, CD90 and CD34 during their leukocyte morphology phase, can be distinguished from HSC by their obligate plastic adherence and the presence of mesenchymal associated markers CD105, CD29, CD73, CD13 and embryonic associated markers SSEA-3 and SSEA-4. Additionally using currently available technology, HSC are unable to be cultured in vitro without further differentiation while MLPC can be expanded for many generations without differentiation. MLPC also differ from MSC and USSC by their more gracile in vitro culture appearance, thread-like cytoplasmic projections and their preference for low density culture conditions for optimal growth.

MLPC or cells having a heptatocyte phenotype also can be characterized based on the expression of one or more genes. Methods for detecting gene expression can include, for example, measuring levels of the mRNA or protein of interest (e.g., by Northern blotting, reverse-transcriptase (RT)-PCR, microarray analysis, Western blotting, ELISA, or immunohistochemical staining). The gene expression profile of MLPC is significantly different than other cell types. Microarray analysis indicated that the MLPC lines have an immature phenotype that differs from the phenotypes of, for example, CD133+ HSC, lineage negative cells (Forraz et al., Stem Cells, 22(1):100-108 (2004)), and MSC (catalog #PT-2501, Cambrex, Walkersville, Md., U.S. Pat. No. 5,486,359), which demonstrate a significant degree of commitment down several lineage pathways. See, e.g., U.S. Pat. Nos. 7,670,596 and 7,622,108

Comparison of the gene expression profile of MLPC and MSC demonstrates MSC are more committed to connective tissue pathways. There are 80 genes up-regulated in MSC, and 152 genes up-regulated in MLPC. In particular, the following genes were up-regulated in MLPC when compared with MSC, i.e., expression was decreased in MSC relative to MLPC: ITGB2, ARHGAP9, CXCR4, INTEGRINB7, PECAM1, PRKCB_1, PRKCB_3, IL7R, AIF1, CD45_EX10-11, PLCG2, CD37, PRKCB_2, TCF2_1, RNF138, EAAT4, EPHA1, RPLPO, PTTG; SERPINA1_2, ITGAX, CD24, F11R, RPL4, ICAM1, LMO2, HMGB2, CD38, RPL7A, BMP3, PTHR2, S100B, OSF, SNCA, GRIK1, HTR4, CHRM1, CDKN2D, HNRPA1, IL6R, MUSLAMR, ICAM2, CSK, ITGA6, MMP9, DNMT1, PAK1, IKKB, TFRC_MIDDLE, CHI3L2, ITGA4, FGF20, NBR2, TNFRSF1B, CEBPA_3, CDO1, NFKB1, GATA2, PDGFRB, ICSBP1, KCNE3, TNNC1, ITGA2B, CCT8, LEFTA, TH, RPS24, HTR1F, TREM1, CCNB2, SELL, CD34, HMGIY, COX7A2, SELE, TNNT2, SEM2, CHEK1, CLCN5, F5, PRKCQ, ITGAL, NCAM2, ZNF257-MGC12518-ZNF92-ZNF43-ZNF273-FLJ90430, CDK1, RPL6, RPL24, IGHA1-IGHA2_M, PUM2, GJA7, HTR7, PTHR1, MAPK14, MSI2_1, KCNJ3, CD133, SYP, TFRC_5PRIME, TDGF1-TDGF3_2, FLT3, HPRT, SEMA4D, ITGAM, KIAA0152_3, ZFP42, SOX20, FLJ21190, CPN2, POU2F2, CASP8_1, CLDN10, TREM2, TERT, OLIG1, EGR2, CD44 EX3-5, CD33, CNTFR, OPN, COL9A1_2, ROBO4, HTR1D_1, IKKA, KIT, NPPA, PRKCH, FGF4, CD68, NUMB, NRG3, SALL2, NOP5, HNF4G, FIBROMODULIN, CD58, CALB1, GJB5, GJA5, POU5F_1, GDF5, POU6F1, CD44 EX16-20, BCAN, PTEN1-PTEN2, AGRIN, ALB, KCNQ4, DPPA5, EPHB2, TGFBR2, and ITGA3. See, e.g., U.S. Pat. Nos. 7,670,596 and 7,622,108. CD106 are positive in MLPC and negative in MSC. Confocal analysis of stem-cell-associated markers SOX-2 and Oct 3/4 show distinctive differences between the MSC and MLPC. MSC are negative for both SOX-2 and Oct 3/4, while MLPC are positive for both.

MLPC express a number of genes associated with “stemness,” which refers to the ability to self-renew undifferentiated and ability to differentiate into a number of different cell types. Genes associated with “stemness” include the genes known to be over-expressed in human embryonic stem cells, including, for example, POU5F (Oct4), TERT, and ZFP42. For example, 65 genes associated with protein synthesis are down-regulated, 18 genes linked with phosphate metabolism are down-regulated, 123 genes regulating proliferation and cell cycling are down-regulated, 12 different gene clusters associated with differentiation surface markers are down-regulated, e.g., genes associated with connective tissue, including integrin alpha-F, laminin and collagen receptor, ASPIC, thrombospondins, endothelium endothelin-1 and -2 precursors, epidermal CRABP-2, and genes associated with adipocytes, including, for example, the leptin receptor, and 80 genes linked to nucleic acid binding and regulation of differentiation are up-regulated. Thus, the immaturity of a population of MLPC can be characterized based on the expression of one or more genes (e.g., one or more of CXCR4, FLT3, TERT, KIT, POU5F, or hematopoietic CD markers such as CD9, CD34, and CD133). See, e.g., U.S. Pat. Nos. 7,670,596 and 7,622,108

### Modified Populations of MLPC

MLPC can be modified such that the cells can produce one or more polypeptides or other therapeutic compounds of interest. To modify the isolated cells such that a polypeptide or other therapeutic compound of interest is produced, the appropriate exogenous nucleic acid must be delivered to the cells. In some embodiments, the cells are transiently transfected, which indicates that the exogenous nucleic acid is episomal (i.e., not integrated into the chromosomal DNA). In other embodiments, the cells are stably transfected, i.e., the exogenous nucleic acid is integrated into the host cell's chromosomal DNA. The term “exogenous” as used herein with reference to a nucleic acid and a particular cell refers to any nucleic acid that does not originate from that particular cell as found in nature. In addition, the term “exogenous” includes a naturally occurring nucleic acid. For example, a nucleic acid encoding a polypeptide that is isolated from a human cell is an exogenous nucleic acid with respect to a second human cell once that nucleic acid is introduced into the second human cell. The exogenous nucleic acid that is delivered typically is part of a vector in which a regulatory element such as a promoter is operably linked to the nucleic acid of interest.

Cells can be engineered using a viral vector such as an adenovirus, adeno-associated virus (AAV), retrovirus, lentivirus, vaccinia virus, measles viruses, herpes viruses, or bovine papilloma virus vector. See, Kay et al. (1997) Proc. Natl. Acad. Sci. USA 94:12744-12746 for a review of viral and non-viral vectors. A vector also can be introduced using mechanical means such as liposomal or chemical mediated uptake of the DNA. For example, a vector can be introduced into an MLPC by methods known in the art, including, for example, transfection, transformation, transduction, electroporation, infection, microinjection, cell fusion, DEAE dextran, calcium phosphate precipitation, liposomes, LIPOFECTIN™, lysosome fusion, synthetic cationic lipids, use of a gene gun or a DNA vector transporter.

A vector can include a nucleic acid that encodes a selectable marker. Non-limiting examples of selectable markers include puromycin, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture.

MLPC also can have a targeted gene modification. Homologous recombination methods for introducing targeted gene modifications are known in the art. To create a homologous recombinant MLPC, a homologous recombination vector can be prepared in which a gene of interest is flanked at its 5′ and 3′ ends by gene sequences that are endogenous to the genome of the targeted cell, to allow for homologous recombination to occur between the gene of interest carried by the vector and the endogenous gene in the genome of the targeted cell. The additional flanking nucleic acid sequences are of sufficient length for successful homologous recombination with the endogenous gene in the genome of the targeted cell. Typically, several kilobases of flanking DNA (both at the 5′ and 3′ ends) are included in the vector. Methods for constructing homologous recombination vectors and homologous recombinant animals from recombinant stem cells are commonly known in the art (see, e.g., Thomas and Capecchi, 1987, Cell 51:503; Bradley, 1991, Curr. Opin. Bio/Technol. 2:823-29; and PCT Publication Nos. WO 90/11354, WO 91/01140, and WO 93/04169.

MLPC can be cryopreserved by suspending the cells (e.g. 5×10⁶ to 2×10⁷ cells/mL) in a cryopreservative such as dimethylsulfoxide (DMSO, typically 1 to 10%) or in fetal bovine serum, human serum, or human serum albumin in combination with one or more of DMSO, trehalose, and dextran. For example, (1) fetal bovine serum containing 10% DMSO; (2) human serum containing 10% DMSO and 1% Dextran; (3) human serum containing 1% DMSO and 5% trehalose; or (4) 20% human serum albumin, 1% DMSO, and 5% trehalose can be used to cryopreserve MLPC. After adding cryopreservative, the cells can be frozen (e.g., to −90° C.). In some embodiments, the cells are frozen at a controlled rate (e.g., controlled electronically or by suspending the cells in a bath of 70% ethanol and placed in the vapor phase of a liquid nitrogen storage tank. When the cells are chilled to −90° C., they can be placed in the liquid phase of the liquid nitrogen storage tank for long term storage. Cryopreservation can allow for long-term storage of these cells for therapeutic use. MLPC have been stored in this fashion for 15 years and retain their viability.

The invention will be further described in the following examples, which do not limit the scope of the invention described in the claims.

### EXAMPLES

### Example 1: Purification of MLPC

The cell separation reagent of Table 1 was used to isolate MLPC from the non-agglutinated supernatant phase. See FIG. 1 for a schematic of the purification.

TABLE 1
Cell Separation Reagent

| | | |
|---|---|---|
| Dextran (average molecular weight 413,000) | 20 | g/l |
| Hank's balanced salt solution (pH 7.2-7.4) | 50 | ml/l |
| Anti-CD15 (murine IgM monoclonal | 0.1-15 | mg/L |

| | |
|---|---|
| antibody, clone 324.3.B9) | (e.g., about 2.0 mg/L) |

Briefly, 50-150 ml of CPDA anti-coagulated umbilical cord blood (<48 hours old) was gently mixed with an equal volume of cell separation composition described in Table 1 for 30 minutes. After mixing was complete, the container holding the blood/cell separation composition mixture was placed in an upright position and the contents allowed to settle by normal 1×g gravity for 30 minutes. After settling was complete, the non-agglutinated cells were collected from the supernatant. The cells were recovered from the supernatant by centrifugation then washed with PBS. Cells were resuspended in complete MSCGM (Mesenchymal stem cell growth medium, catalog #PT-3001, Cambrex, Walkersville, Md.) and adjusted to 2-9×10⁶ cells/ml with complete MSCGM. Cells were plated in a standard plastic tissue culture flask (e.g., Corning), chambered slide, or other culture device and allowed to incubate overnight at 37° C. in a 5% CO₂ humidified atmosphere. All subsequent incubations were performed at 37° C. in a 5% CO₂ humidified atmosphere unless otherwise noted. MLPC attached to the plastic during this initial incubation. Non-adherent cells (T-cells, NK-cells and CD34+ hematopoietic stem cells) were removed by vigorous washing of the flask or well with complete MSCGM.

MLPC cultures were fed periodically by removal of the complete MSCGM and addition of fresh complete MSCGM. Cells were maintained at concentrations of 1×10⁵-1×10⁶ cells/75 cm² by this method. When cell cultures reached a concentration of 8×10⁵-1×10⁶ cells/75 cm², cells were cryopreserved using 10% DMSO and 90% serum or expanded into new flasks. Cells were recovered from the adherent cultures by removal of the complete MSCGM and replacement with Tryp-LE (Gibco). Cells were incubated for 15-60 minutes at 37° C. then collected from the flask and washed in complete MSCGM. Cells were then replated at 1×10⁵ cells/mL. Cultures that were allowed to achieve confluency were found to have diminished capacity for both proliferation and differentiation. Subsequent to this finding, cultures were not allowed to achieve higher densities than 5×10⁶ cells/75 cm².

### Example 2: Clonal MLPC Cell Lines

After the second passage of MLPC cultures from Example 1, the cells were detached from the plastic surface of the culture vessel by substituting Tryp-LE (pH 7.3) for the cell culture medium. The cells were diluted to a concentration of 1.3 cells/ml in complete MSCGM and distributed into a 96 well culture plate at a volume of 0.2 ml/well, resulting in an average distribution of approximately 1 cell/3 wells. After allowing the cells to attach to the plate by overnight incubation at 37° C., the plate was scored for actual distribution. Only the wells with 1 cell/well were followed for growth. As the cells multiplied and achieved concentrations of 1-5×10⁵ cells/75 cm², they were transferred to a larger culture vessel in order to maintain the cells at a concentration between 1×10⁵ and 5×10⁵ cells/75 cm² to maintain logarithmic growth. Cells were cultured at 37° C. in a 5% CO₂ atmosphere.

Efficiency of cloning suggested that only 5-10% of the CD45⁻, CD9⁺, CD105⁺, CD106⁺, CD90⁺ were capable of clonability, and ability to be expanded to master and working cell banks. Some clones were capable of >60 population doublings before scenescence. See, e.g., U.S. Pat. Nos. 7,670,596 and 7,622,108

Immortalization of MLPC by the Insertion of the TERT Gene

pRRLsin.hCMV hTERT lentiviral expression plasmid (Dr. Noriyuki Kasahara, Dept of Medicine, UCLA, CA) was used in this experiment. The telomerase vector was produced by three plasmid transient transductions using 10 μg of the self-inactivating (sin) hTERT lentiviral expression plasmid, 10 μg of the gag/pol plasmid, pCMV delta 8.2, and 2 μg of the envelope plasmid, pCMV VSVG, in a calcium phosphate transduction protocol according to the manufacturer's directions (Clontech). HEK 293T cells at 60-70% confluence in a 10 cm dish were given 10 ml fresh medium (DMEM, 10% FBS without antibiotics) 3-4 hours prior to transduction. After incubation of transduced cells overnight at 37° C., 5% CO₂, the medium was replaced with 6 ml fresh medium and incubated for an additional 24 hours. The supernatant was then collected and passed through a 0.45 μm filter and stored at −80° C. until used for transduction.

Mixed MLPC cultures (passage 12) were seeded at a density of 5×10⁴ cells/well of a 6-well tissue culture dish in complete MSCGM (without antibiotics) 24-hours prior to transduction. The vector supernatant was diluted 1:10 with DMEM 10% FBS containing 8 μg/ml polybrene and 1 ml was added to each well from which the growth medium had been removed. After 4 hours at 37° C., 5% CO₂, the diluted vector was removed and replaced with MSCGM. To get an estimate of transduction efficiency, MLPC were also transduced identically in parallel with pRRL sinhCMV GFP vector supernatants which had been serially diluted. GFP expression was analyzed 60 hours after transduction using FACS analysis. The titer of the GFP vector supernatant was 5×10⁴.

Establishment of MLPC-TERT Cell Lines

As with the non-transfected MLPC, clonal MLPC-TERT cell lines were developed by limited dilution cloning. Wells with only one detectable cell were propagated in larger culture vessels to achieve cell numbers sufficient for analysis. Of the ten stable cell lines that were developed, one clone exhibited the combined characteristics of immortality and differentiation outside of mesodermal outcomes, E12-TERT (E12). This cell line has been in culture for multiple years and was used in some of the experiments described herein.

### Example 3: Differentiation of MLPC to Hepatocyte-Like Cells

The protocol for differentiation of MLPC and MLPC-derivative cells is presented schematically in FIG. 2. The differentiation procedure entails a phenotypic transition from undifferentiated MLPC to committed endodermal cell to hepatocyte/pancreatic precursor to committed hepatocyte precursor to fully mature hepatocyte-like cell.

MLPC were seeded onto collagen-coated culture vessels at a concentration of 2.5×10⁴ cells/cm² surface area in media formulation 1 shown in Table 2 and allowed to attach to the substrate overnight and achieve fibroblast-like morphology.

TABLE 2
Medium 1: MLPC
Plating Medium

| | |
|---|---|
| Williams Medium E | 500 ml |
| Fetal Bovine Serum | 50 ml |
| Glutamax | 5 ml |
| Penicillin/Streptomycin | 5 ml |

After establishment of MLPC cultures (usually 1-2 days) medium 1 was removed and replaced with Medium 2 shown in Table 3 and cultured for 5-7 days. Medium 2 is replaced at day 5-7 of culture with fresh Medium 2. After this culture period, the MLPC have been differentiated to the stage of committed endodermal cell. The resultant cells express markers consistent with committed endodermal cells, including SOX-17.

TABLE 3
Medium 2: Endodermal Induction Medium

| | |
|---|---|
| Williams Medium E | 500 ml |
| Fetal Bovine Serum | 50 ml |
| Glutamax | 5 ml |
| Penicillin/Streptomycin | 5 ml |
| ITS solution | 5 ml |
| Activin A | 100 ng/ml |

At the conclusion of culture in Medium 2, media was exchanged to Medium 3 shown in Table 4. Cells were cultured in Medium 3 for 14-21 days, with culture media changes 3 times weekly. After 7-10 days of culture, the cells have differentiated to a committed hepato-pancreatic cell. Cells at this stage express cell markers common to both hepatocytes and pancreatic cells, including but not limited to SOX-17, GATA-4, and alkaline phosphatase. Cells at this stage can be differentiated towards pancreatic lineage by removal of Medium 3 and replacement with a pancreatic differentiation medium differing from Medium 3.

After a further culture of 7-14 days in Medium 3, the cells progress to a committed hepatocyte precursor. These cells express various markers associated with commitment to the hepatocyte lineage including but not limited to alkaline phosphatase, alpha fetoprotein, c-reactive protein, hepatocyte growth factor receptor, nestin and SOX-17, and asialo glycoprotein receptor 1.

TABLE 4
Medium 3: Hepatocyte Induction Medium

| | |
|---|---|
| Williams Medium E | 500 ml |
| Fatty acid-free Bovine Serum Albumin | 50 ml |
| Glutamax | 5 ml |
| Penicillin/Streptomycin | 5 ml |
| Hydrocortisone-21-hemisuccinate | 5 mM |
| ITS supplement | 5 ml |
| Epithelial growth factor | 80 ng/ml |
| Fibroblast growth factor basic | 20 ng/ml |
| Fibroblast growth factor 4 | 20 ng/ml |
| Hepatocyte growth factor | 40 ng/ml |
| Stem cell factor | 40 ng/ml |
| Oncostatin M | 20 ng/ml |
| Bone morphogenic protein 4 | 20 ng/ml |
| Interleukin 1 beta | 10 ng/ml |

After the cells have achieved committed hepatocyte precursor morphology and phenotype, Medium 3 was removed and exchanged with Medium 4. The composition of Medium 4 is shown in Table 5. Cells were cultured for an additional 7-10 days whereby the cells achieved the morphology and phenotype of cells consistent with mature primary hepatocytes.

TABLE 5
Medium 4: Hepatocyte Maturation Medium

| | |
|---|---|
| Williams Medium E | 500 ml |
| Fatty acid-free Bovine Serum Albumin | 50 ml |
| Glutamax | 5 ml |
| Penicillin/Streptomycin | 5 ml |
| Hydrocortisone-21-hemisuccinate | 5 mM |
| ITS supplement | 5 ml |
| Epithelial growth factor | 80 ng/ml |
| Fibroblast growth factor basic | 20 ng/ml |
| Fibroblast growth factor 4 | 20 ng/ml |
| Hepatocyte growth factor | 40 ng/ml |
| Stem cell factor | 40 ng/ml |
| Oncostatin M | 20 ng/ml |
| Bone morphogenic protein 4 | 20 ng/ml |
| Interleukin 1 beta | 10 ng/ml |
| DMSO | 0.5% |
| Retinoic acid | 30 μg/ml |

Phenotypic expression of hepatocyte-associated markers assayed by immunofluorescent confocal microscopy of MLPC at various stages of differentiation is shown in Table 10.

### Example 4: Culture, Expansion, and Differentiation of Marmoset Embryonic Stem Cells (M-ESC)

M-ESC (common marmoset (Callithrix jacchus) embryonic stem cell line cj367) were seeded into a T-75 flask at a concentration of 4×10⁶ cells in 13 ml of Williams E medium supplemented with 10% fetal bovine serum and allowed to adhere overnight. At that time the medium was changed to medium A (Table 6) and cultured for 5-7 days with a single medium change at day 3. After this, the medium was exchanged for medium B (Table 7) and cultured for another 14 days, with 6 medium changes over that time. The medium was then exchanged with the final medium C (Table 8) where the cells matured to hepatocyte-like cells. Cells achieving final status can be further propagated in the medium of Table 4.

TABLE 6
Medium A: ESC Maintenance and Expansion Medium

| | |
|---|---|
| E8 Media | 500 ml |
| E8 Supplement | 10 ml |
| Glutamax | 5 ml |
| Lipid Concentrate | 5 ml |
| Nodal | 100 pg/ml |
| Glutathione | 1.94 ng/ml |

TABLE 7
Medium B: Endodermal Induction Medium

| | |
|---|---|
| RPMI-1640 | 500 ml |
| Fetal Bovine Serum | 50 ml |
| Activin A | 100 ng/ml |
| FGF-2 | 5 ml |

TABLE 8
Medium C: Hepatocyte Differentiation Medium

| | | |
|---|---|---|
| RPMI-1640 | 500 | ml |
| Fetal Bovine Serum | 50 | ml |
| FGF-2 | 10 | ng/ml |
| BMP-4 | 10 | ng · ml |
| HGF | 20 | ng/ml |

TABLE 9

| Medium D: Hepatocyte Maturation Medium | | |
|---|---|---|
| RPMI-1640 | 500 | ml |
| Fetal Bovine Serum | 50 | ml |
| Oncostatin M | 20 | ng/ml |

### Example 5: Immunofluorescent Confocal Microscopy

Cryo-preserved primary human hepatocytes were obtained from Zenotech (Kansas City, Kans.). Cells were thawed with OptiThaw medium and enumerated with OptiCount medium in a standard hemacytometer. Cells were diluted to a final concentration of 106 cells/ml of Opti-Plate medium. Cells were plated in collagen-coated 6 well plates (BD Biosciences) at 1 ml per well. After 4 hours of plating, the medium was changed to OptiCulture medium for the duration of culture. Medium was exchanged every 24 hours.

Cells to be analyzed (MLPC in different media, M-ESC, hepatocyte-like cells derived from M-ESC (ESC-H), and primary human hepatocytes that were harvested from their culture vessels by dissociation with Tryp-LE (12605-028, Life Technologies, Grand Island, N.Y.). Cells were enumerated and resuspended in the same medium they had just been cultured in, at a density of 10⁵ cells/ml. Two hundred μl of cell suspension was added to each well of a collagen-coated 16-well glass chamber slide. Cells were cultured overnight to facilitate adherence to the slide. After attachment, cells were fixed for 1 hour in 1% formalin. After fixation, cells were permeabilized by PermaCyte Medium (BioE). Cells were incubated with approximately 100 ng of antibodies specific for alkaline phosphatase, alpha fetoprotein, albumin, c-reactive protein, hepatocyte growth factor receptor, coagulation factor VII, coagulation factor IX, nestin, SOX-17, P450 CYP 1A2, P450 CYP 3A4, asialo-glycoprotein receptor 1, hepatocyte nuclear factor-4, GATA-4 and alpha-1-antitrypsin for 40 minutes. Cells were then washed with PermaCyte to remove unbound antibody. Cells then were stained with secondary antibodies specific for mouse or rabbit antibody labeled with either Alexa 488 or Alexa 594 dyes. Cells were counterstained with DAPI dye to visualize the nucleus of each cell. Positivity of staining was confirmed by comparison to cells stained with antibody isotype controls. Comparative results of the confocal microscopy is shown in Table 10.

Similar experiments were repeated with control E12 MLPC cells and cells at various stages of differentiation (committed endoderm, hepatocyte precursor, mature hepatocyte like cells (mature HLC), and primary hepatocytes (PH)). To analyze the expression of hepatocyte-associated markers, cells were fixed and permeabilized as described above, and incubated for 40 minutes with approximately 100 ng of monoclonal antibodies (from R&D Systems (Minneapolis, Minn.) unless indicated otherwise) specific for alkaline phosphatase (MAB1448), α-fetoprotein (MAB1369), albumin (MAB1456), c-reactive protein (MAB17071), hepatocyte growth factor receptor (MAB3583), nestin (MAB1269), SOX-17 (MAB1924), asialoglycoprotein receptor 1 (MAB4394), hepatocyte nuclear factor-4 (ABIN561308), GATA-4, α-1-antitrypsin, cytokeratin19 (MAB3608), SOX-2 (AF2018), SOX-9 (AF2018), EpCAM (MAB9601), Oct 3/4 (MAB1759), coagulation factor VII (MA5-16932), coagulation factor IX (HYB133-01-02) (from Invitrogen, Rockford, Ill.), P450 CYP 1A2 (ab151728), P450 CYP 3A4 (ab124921), glucuronosyltransferase isoforms UGT1A1 and UGT2B7 (ab126269 and ab194697 from Abcam, Cambridge, Mass.), and TERT (NB100-317 from Novus, Littleton, Colo.) to label the cells. Unbound antibody was removed by washing with PermaCyte and the cells were counterstained with secondary antibodies specific for mouse (A-11005), rabbit (A-11072), rat (A-11007) or goat (A-11080) antibody labelled with Alexa 594 dye (Life Technologies, (Eugene, Oreg.)). The nucleus of the cells was visualized by staining with DAPI. Marker expression was confirmed by positive staining when compared to cells stained with antibody isotype controls (QTC1000, CMDG, St. Paul, Minn.) analyzed using the Olympus Fluoview 1000 confocal microscope. Comparative results of the confocal microscopy is shown in Table 11.

By immunohistochemistry, the differentiation of MLPC to committed endodermal cells was characterized by the expression of GATA4 and SOX17 and lack of expression of the more hepatocytes-specific markers. These included AFP, albumin, ASGr1, HNF4, HGFr, A1AT, CYP1A2, CYP 3A4, UGT1A1, and UGT2B7. Interestingly, SOX9, CK19, and EpCAM were expressed in undifferentiated MLPC and were maintained throughout the differentiation process. Further differentiation of the MLPC to the committed hepatocyte precursor cell was characterized by the expression of more hepatocyte-specific markers such as α-fetoprotein, albumin, ASGr1, HNF4, HGFr, A1AT, CYP 1A2, CYP 3A4, UGT1A1 and UGT2B7. Notably, HNF4 was expressed only cytoplasmically in the committed hepatocyte precursor cells, but was also detected within the nucleus of fully mature hepatocyte-like cells. Not surprisingly, HNF4 was also identified in the nucleus of PH. All markers expressed by PH were also expressed by the fully mature hepatocyte-like cells.

TABLE 10
Comparative Table of Hepatocyte-specific Marker Expression Identified by Immunofluorescent Confocal Microscopy

| | | Medium 1 | Medium 2 | Medium 3 | | | Primary Human |
|---|---|---|---|---|---|---|---|
| Analyte | MLPC | (Table 2) | (Table 3) | (Table 4) | M-ESC | ECS-H | Hepatocytes |
| Alkaline Phosphatase | Neg | Neg | Pos | Pos | Neg | Pos | Pos |
| Alpha fetoprotein | Neg | Neg | Pos | Pos | Neg | Pos | Pos |
| Albumin | Neg | Neg | Pos | Pos | Neg | Pos | Pos |
| C-reactive protein | Neg | Pos | Pos | Pos | Neg | Pos | Pos |
| Hepatocyte Growth | Neg | Neg | Pos | Pos | Neg | Pos | Pos |
| Factor Receptor | | | | | | | |
| Complement Factor | Neg | Pos | Pos | Pos | Neg | Pos | Pos |
| VII | | | | | | | |
| Complement Factor | Neg | Neg | Neg | Pos | Neg | Neg | Pos |
| IX | | | | | | | |
| SOX 17 | Neg | Neg | Pos | Pos | Neg | Pos | Pos |
| P450 CYP 3A4 | Neg | Neg | Pos | Pos | Neg | Pos | Pos |
| P450 CYP 1A2 | Neg | Neg | Pos | Pos | Neg | Pos | Pos |
| Asialo glycoprotein | Neg | Neg | Pos | Pos | Neg | Pos | Pos |
| receptor 1 | | | | | | | |
| Hepatocyte Nuclear | Neg | Neg | cytoplasmic | cytoplasmic | Neg | cytoplasmic | cytoplasmic |
| Factor 4 | | | | and nuclear | | and nuclear | and nuclear |
| GATA-4 | Neg | Neg | Pos | Pos | Neg | Pos | Pos |
| Alpha-1-antitrypsin | Neg | Neg | Pos | Pos | Neg | Pos | Pos |

TABLE 11
Comparative Table of Hepatocyte-specific Marker Expression Identified by Immunofluorescent Confocal Microscopy

| | E12 | Committed | Hepatocyte | | |
|---|---|---|---|---|---|
| Analyte | MLPC | Endoderm | Precursor | Mature HLC | PH |
| Alkaline Phosphatase | Neg | Neg | Pos | Pos | Pos |
| Alpha Fetoprotein | Neg | Neg | Pos | Pos | Pos |
| Albumin | Neg | Neg | Pos | Pos | Pos |
| C-reactive Protein | Neg | Pos | Pos | Pos | Pos |
| Hepatocyte Growth Factor Receptor | Neg | Neg | Pos | Pos | Pos |
| Coagulation Factor VII | Neg | Neg | Neg | Pos | Pos |
| Coagulation Factor IX | Neg | Neg | Neg | Pos | Pos |
| Nestin | Neg | Neg | Pos | Pos | Pos |
| SOX 17 | Neg | Pos | Pos | Pos | Pos |
| P450 CYP 3A4 | Neg | Neg | Pos | Pos | Pos |
| P450 CYP 1A2 | Neg | Neg | Pos | Pos | Pos |
| Asialoglycoprotein Receptor 1 | Neg | Neg | Pos | Pos | Pos |
| Hepatocyte Nuclear Factor 4 | Neg | Neg | Cytoplasmic | Cytoplasmic | Cytoplasmic |
| | | | Positive, | Positive, | Positive, |
| | | | Nuclear | Nuclear | Nuclear |
| | | | Negative | Positive | Positive |
| GATA-4 | Neg | Pos | Pos | Pos | Pos |
| Alpha-1-Antitrypsin | Neg | Neg | Pos | Pos | Pos |
| SOX2 | Pos | Pos | Pos | Pos | Pos |
| SOX9 | Pos | Pos | Pos | Pos | Pos |
| CK19 | Pos | Pos | Pos | Pos | Pos |
| EpCAM | Pos | Weak Pos | Pos | Pos | Pos |
| UGT1A1 | Neg | Neg | Pos | Pos | Pos |
| UGT2B7 | Neg | Neg | Pos | Pos | Pos |
| TERT | Pos | Pos | Pos | Pos | Weak in |
| | | | | | some cells |

### Example 6: Expansion and Long-term Culture of Hepatocyte-differentiated

Stem Cells

After differentiation of marmoset ESC, MLPC and TERT-MLPC to hepatocyte-like cells, the cells were cultured further to determine the longevity of the differentiated cells and the maintenance of the hepatocyte-like phenotypes. The formulation of the medium utilized for expansion and longevity testing is shown in Table 4 (medium 3). For each of the different cell types, 10,000 cells were seeded into each well of a six well plate with 2 ml of the media from Table 4. Cells were allowed to expand to 5×10⁵ cells and the time of the expansion was recorded. It was determined that in the Table 4 medium, marmoset ESC-hepatocytes could double their population in an average of 39 hours and be continually cultured for 2 months. MLPC-hepatocytes could double their population in an average of 35 hours and could be cultured for 2 months before senescence. TERT-MLPC-hepatocytes could double their population in an average of 23 hours and have been serially cultured for 6 months.

Karyotype analysis was performed on the cell populations. Adherent marmoset ESCs were harvested after overnight colcemid arrest. The cells were treated with 0.75 M KCl hypotonic solution and fixed with 3:1 methanol:acetic acid. They were then spread onto glass slides according to standard cytogenetic protocols and stained with Wright-Giemsa stain. Twenty G-banded metaphases were analyzed using an Olympus BX61 microscope outfitted with 10× and 100× objectives. Metaphase chromosomes were imaged and karyotyped using Applied Spectral Imaging (ASI) software. Activin A treatment did not alter the chromosomes of ESCs. Undifferentiated ESCs, activin A-treated ESCs and differentiated HLCs dis-275 played normal female karyotype (46, XX), which was similar to published data on marmoset ESC cell lines.

### Example 7: Production of Albumin

Albumin production was analyzed in E12 MLPC, differentiated hepatocyte like cells (HLC), E12/PH fusion cells, and PH (Zenotech), with an enzymatic ELISA assay as described above. Cells were cultured for 3 days in the medium specific for each cell type, after which they were dissociated from the culture plate using the Tryp-LE reagent, counted with a hemocytometer and pelleted at 400×g for 5 minutes. Supernatant was discarded and cells were re-suspended in 1 ml of WIF water (Millipore/Sigma). Cells then were subjected to three repeated freeze-thaw cycles of freezing at −80° C. and thawing at room temperature to rupture and release the intracellular contents into the fluid phase. After the last freeze-thaw cycle, cells were centrifuged at 1000×g for 10 minutes to pellet the cell debris and the supernatant from the cell lysate was collected and analyzed by the albumin ELISA (Abcam, Cambridge, Mass.), according to the manufacturer's instructions. Results were standardized to 1×10⁶ cells/ml of supernatant to enable comparison between cell culture samples with different cell numbers. Resultant samples were analyzed on the Emax microplate reader (Molecular Devices. San Jose, Calif.) and processed with SoftMax PRO 4.8 Analysis Software. E12 MLPC and PHs were used as negative and positive controls, respectively. A total of four separate determinations were carried on different days. The results are shown in Table 12, and expressed as pg/mL per 10⁶ cells. Both differentiated MLPC and fusion E12/PH cells were capable of producing albumin.

TABLE 12
Albumin Production

| E12 MLPC | Differentiated HLC | E12/PH Fusion | PH |
|---|---|---|---|
| 0.71 ± 0.92 | 1495.27 ± 427.22 | 5706.9 ± 4845.52 | 15725.38 ± 5490.89 |

### Example 8: Production of Urea

A critical function of hepatocytes is to convert toxic ammonia and uric acid to urea for excretion. Urea production was analyzed in E12 MLPC, differentiated hepatocyte like cells (HLC), primary hepatocytes (Zenotech), and E12/PH hepatocyte fusion cells. Cells were cultured for 3 days in the medium specific for each cell type. After three days, cells were dissociated from the culture plate using the Tryp-LE reagent, counted with a hemocytometer and centrifuged at 400×g for 5 minutes to pellet them. The supernatant was discarded and cells were re-suspended in 1 ml of WIF water (Millipore/Sigma). Cells were then exposed to three repeated freeze-thaw cycles of freezing at −80° C. and thawing at room temperature to rupture cell membranes and release contents into the fluid phase. After the last freeze-thaw cycle, cells were centrifuged at 1000×g for 10 minutes to pellet cell debris, and the supernatant from the cell lysate was collected and analyzed with a colorimetric assay (Biovision, Milpitas, Calif.) urea assay, according to the manufacturer's instructions. Results were standardized to 1×10⁶ cells/ml of supernatant to enable comparison between cell culture samples with different cell numbers. Resultant samples were analyzed on the Emax microplate reader (Molecular Devices. San Jose, Calif.) and processed with SoftMax PRO 4.8 Analysis Software. E12 MLPC and primary hepatocytes (PH) were used as negative and positive controls, respectively. At least four separate determinations were carried on different days. The results are shown in Table 12, and expressed as nmoles/mL per 10⁶ cells. Both differentiated MLPC and fusion E12/PH cells both express urea in concentrations comparable or superior to the primary hepatocytes. Results are compared within a single assay, and the results accurately portray the relative results within the assay.

TABLE 13
Urea Production

| E12 MLPC | Differentiated HLC | PH |
|---|---|---|
| 1.55 ± 0.4 | 11.09 ± 0.96 | 8.28 ± 2.73 |
| E12 MLPC | E12/PH Fusion Cells | PH |
| 5.72 ± 5.61 | 78.48 ± 24.11 | 28.72 ± 22.9 |

### Example 10—RT-PCR Analysis

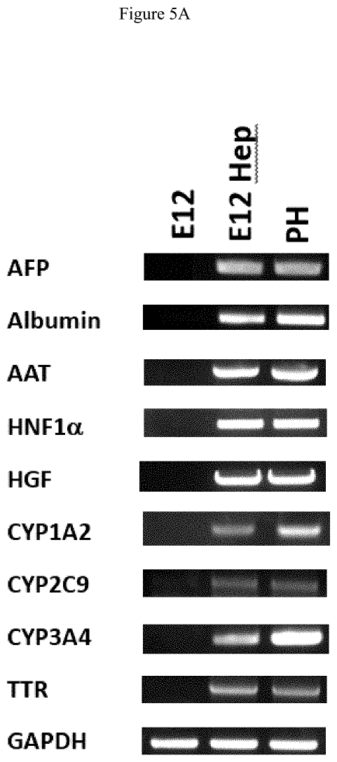

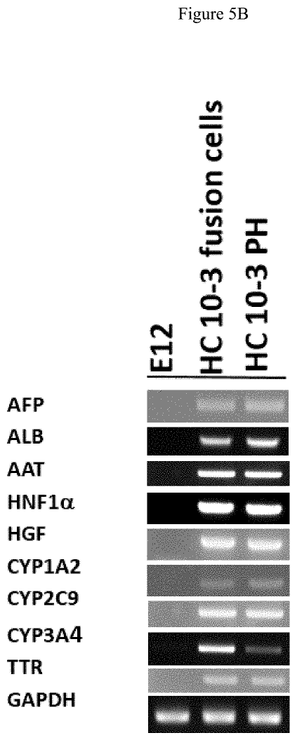

Expression of hepatocyte-specific genes, including alpha-fetoprotein (AFP), α-1-antitrypsin (AAT), transthyretin (TTR), cytochrome P450 1A2 (CYP 1A2), cytochrome P450 3A4 (CYP 3A4), cytochrome P450 2C9 (CYP 2C9), hepatocyte nuclear factor 1α (HNF1A), hepatocyte growth factor (HGF), albumin (ALB), and housekeeping gene glyceraldehyde-3-phosphate dehydrogenase (GAPDH), was assessed in E12 MLPC, hepatocyte like cells differentiated from MLPC (E12-Hep), primary hepatocytes (PH), PH from donor HC 10-3, and from fused E12-MLPC to HC 10-3 PH. Total RNA was extracted from the various cell populations using the UltraPure™ Phenol:Chloroform:Isoamyl Alcohol reagent (Invitrogen) and the Platinum® Quantitative RT-PCR ThermoScript™ One-Step System (Invitrogen) was used to carry out quantitative RT-PCR reactions according to manufacturer's recommendations. Total RNA isolated from E12 MLPC was used as the negative control, and that isolated from primary hepatocytes (PH) was used as a positive control. The primers for each of the hepatocyte-specific genes is shown in Table 14. Conditions for PCR reactions were initial denaturation at 94° C. for 3 min followed by 30 cycles of denaturation at 94° C. for 1 min, annealing for 1 min at 56° C., and elongation for 1 min at 72° C. PCR products were then resolved using a 1% agarose gel, and visualized under UV light. As shown in FIGS. 5A and 5B, E12 MLPC were negative for the hepatocyte-specific markers, and both differentiated MLPC and fusion cells show similar levels of expression of hepatocyte-specific genes as primary hepatocytes (PH).

TABLE 14
Primers used for RT-PCR Analysis

| Gene | | SEQ |
|---|---|---|
| Name | Primer Sequences | ID |
| AFP | F: 5′-TGCAGCCAAAGTGAAGAGGGAAGA-3′ | 1 |
| | R: 5′-CATAGCGAGCAGCCCAAAGAAGAA-3′ | 2 |
| AAT | F: 5′-ACTGTCAACTTCGGGGACAC-3′ | 3 |
| | R: 5′-CATGCCTAAACGCTTCATCA-3′ | 4 |
| TTR | F: 5′-TCATCGTCTGCTCCTCCTCT-3′ | 5 |
| | R: 5′-AGGTGTCATCAGCAGCCTTT-3′ | 6 |
| CYP1A2 | F: 5′-CAATCAGGTGGTGGTGTCAG-3′ | 7 |
| | R: 5′-GCTCCTGGACTGTTTTCTGC-3′ | 8 |
| CYP3A4 | F: 5′-AAGTCGCCTCGAAGATACACA-3′ | 9 |
| | R: 5′-AAGGAGAGAACACTGCTCGTG-3′ | 10 |
| CYP2C9 | F: 5′-GGACAGAGACGACAAGCACA-3′ | 11 |
| | R: 5′-CATCTGTGTAGGGCATGTGG-3′ | 12 |
| HNF1A | F: 5′-TACACCACTCTGGCAGCCACACT-3′ | 13 |
| | R: 5′-CGGTGGGTACATTGGTGACAGAAC-3′ | 14 |
| GAPDH | F: 5′-GCACCGTCAAGGCTGAGAAC-3′ | 15 |
| | R: 5′-ATGGTGGTGAAGACGCCAGT-3′ | 16 |
| HGF | F: 5′-GTAAATGGGATTCCAACACGAACAA-3′ | 17 |
| | R: 5′-TGTCGTGCAGTAAGAACCCAACTC-3′ | 18 |

The treatment for terminal liver disease is a liver transplant. This therapeutic treatment is limited by the availability of transplantable livers. Development of future drug-based therapies is dependent upon the availability of PHs to study potential therapies in vitro prior to testing in animal models and then in human clinical trials. Additionally, the effects of drugs developed for the treatment of other non-liver diseases are tested for their effects on liver function initially by in vitro toxicology testing on PHs. A potential bridge to transplantation for liver failure could also involve the availability of functional hepatocytes or hepatocyte-like cells incorporated into an extra-corporeal device containing those cells. Both of these needs are constrained by the lack of donor livers for both transplantation and research. Livers that are deemed unsuitable for transplantation are used as the sources for isolated primary hepatocytes for in vitro drug development and toxicology studies. This results in using cells that may have different capacities with regards to their biological functions. Because of these limitations other methods have been developed to attempt to generate cells with the functional characteristics of well-differentiated hepatocytes and the proliferative capacity to support large-scale production.

The results described herein provide a methodology to differentiate MLPC cell line such as the E12 cell line that immortalized by the insertion of hTERT gene, into cells that express morphology, protein marker, RNA expression and urea production associated with mature hepatocytes. After differentiation, the resultant hepatocyte-like cells retain the immortality and proliferative capacity of the undifferentiated E12 cells.

Differentiation protocols described in earlier studies with MSC did not result in functional, fully mature hepatocyte-like cells when applied to MLPC. As described herein, it was found that an initial commitment to definitive endoderm mediated by activin A was a necessary first step in differentiation. The second step to a committed hepatocyte precursor required the addition of Stem cell factor, bone morphogenic protein 4 (BMP-4), and IL-1β in addition to other factors. The final differentiation to mature hepatocyte-like cells required the addition of DMSO and retinoic acid. The results described herein used an immortalized MLPC cell line such as E12 that is capable of long-term survival in culture and the ability to be expanded to industrial scale quantities while conserving their hepatocyte-like characteristics. These cells could provide a stable repeatable cell standard for the study of liver function, toxicology testing, and a tool for the development of new therapies for liver disease. These cells could also help develop the methods needed to develop artificial liver support systems as a possible bridge to transplant.

### OTHER EMBODIMENTS

The claims may suitably comprise, consist of, or consist essentially of, or be substantially free or free of any of the disclosed or recited elements. The claimed technology is illustratively disclosed herein can also be suitably practiced in the absence of any element which is not specifically disclosed herein. The various embodiments described above are provided by way of illustration only and should not be construed to limit the claims attached hereto. Various modifications and changes may be made without following the example embodiments and applications illustrated and described herein, and without departing from the true spirit and scope of the following claims.

What is claimed is:

1. A composition comprising a culture medium, said medium comprising hydrocortisone, bovine serum albumin, insulin, transferrin, selenium, epithelial growth factor, basic fibroblast growth factor, fibroblast growth factor 4, hepatocyte growth factor, stem cell factor, oncostatin M, bone morphogenic protein 4, and interleukin 1 beta.

2. The composition of claim 1, wherein said culture medium is effective for inducing differentiation of human fetal blood multi-lineage progenitor cells (MLPC) or a clonal line of human fetal blood MLPC to cells having a hepatocyte phenotype

3. The composition of claim 1, wherein said culture medium is effective in the long-term growth and maintenance of primate embryonic stem cell-derived hepatocyte-like cells.

4. The composition of claim 1, said culture medium further comprising an antibiotic.

5. The composition of claim 1, said composition further comprising at least one antibody, said at least one antibody having binding affinity for alkaline phosphatase, alpha fetoprotein, albumin, c-reactive protein, hepatocyte growth factor receptor, complement factor VII, complement factor IX, nestin, SOX-17, P450 CYP 1A2, P450 CYP 3A4, asialo-glycoprotein receptor 1, hepatocyte nuclear factor-4, GATA-4, or alpha-1-antitrypsin.

6. A method of producing a population of cells having a hepatocyte phenotype, said method comprising:
a) providing a collagen-coated culturing device housing a purified population of MLPC or a clonal line of MLPC;
b) culturing said purified population of MLPC or said clonal line of MLPC with a differentiation medium containing Activin A, until cells having an endodermal precursor phenotype are obtained,
c) further culturing said cells having said endodermal precursor phenotype in a differentiation medium comprising hydrocortisone, bovine serum albumin, insulin, transferrin, selenium, epithelial growth factor, basic fibroblast growth factor, fibroblast growth factor 4, hepatocyte growth factor, stem cell factor, oncostatin M, bone morphogenic protein 4 and interleukin 1 beta, to obtain cells having said committed hepatocyte precursor cell phenotype, and
d) further culturing said cells in said differentiation medium in the presence of DMSO and retinoic acid to obtain cells having said hepatocyte phenotype.

7. The method of claim 6, further comprising testing said cells having the hepatocyte phenotype for one or more of alkaline phosphatase, alpha fetoprotein, albumin, c-reactive protein, hepatocyte growth factor receptor, complement factor VII, complement factor IX, nestin, SOX-17, P450 CYP 1A2, P450 CYP 3A4, asialo-glycoprotein receptor 1, hepatocyte nuclear factor-4, GATA-4 and alpha-1-antitrypsin.

8. The method of claim 7, wherein said cells are tested for two or more of alkaline phosphatase, alpha fetoprotein, albumin, c-reactive protein, hepatocyte growth factor receptor, complement factor VII, complement factor IX, nestin, SOX-17, P450 CYP 1A2, P450 CYP 3A4, asialo-glycoprotein receptor 1, hepatocyte nuclear factor-4, GATA-4 and alpha-1-antitrypsin.

9. The method of claim 7, wherein said cells are tested for three or more of alkaline phosphatase, alpha fetoprotein, albumin, c-reactive protein, hepatocyte growth factor receptor, complement factor VII, complement factor IX, nestin, SOX-17, P450 CYP 1A2, P450 CYP 3A4, asialo-glycoprotein receptor 1, hepatocyte nuclear factor-4, GATA-4 and alpha-1-antitrypsin.

10. The method of claim 7, wherein said cells are tested for five or more of alkaline phosphatase, alpha fetoprotein, albumin, c-reactive protein, hepatocyte growth factor receptor, complement factor VII, complement factor IX, nestin, SOX-17, P450 CYP 1A2, P450 CYP 3A4, asialo-glycoprotein receptor 1, hepatocyte nuclear factor-4, GATA-4 and alpha-1-antitrypsin.

11. The method of claim 7, wherein said cells are tested for 10 or more of alkaline phosphatase, alpha fetoprotein, albumin, c-reactive protein, hepatocyte growth factor receptor, complement factor VII, complement factor IX, nestin, SOX-17, P450 CYP 1A2, P450 CYP 3A4, asialo-glycoprotein receptor 1, hepatocyte nuclear factor-4, GATA-4 and alpha-1-antitrypsin.

12. The method of claim 7, wherein said cells are tested for each of alkaline phosphatase, alpha fetoprotein, albumin, c-reactive protein, hepatocyte growth factor receptor, complement factor VII, complement factor IX, nestin, SOX-17, P450 CYP 1A2, P450 CYP 3A4, asialo-glycoprotein receptor 1, hepatocyte nuclear factor-4, GATA-4 and alpha-1-antitrypsin.

13. The method of claim 6, wherein said clonal line of MLPC are immortalized multi-lineage progenitor cells comprising a nucleic acid encoding a telomerase reverse transcriptase.

14. A method of expanding a population of primate embryonic stem cell-derived hepatocyte-like cells, said method comprising growing said cells in a differentiation medium comprising hydrocortisone, bovine serum albumin, insulin, transferrin, selenium, epithelial growth factor, basic fibroblast growth factor, fibroblast growth factor 4, hepatocyte growth factor, stem cell factor, oncostatin M, bone morphogenic protein 4 and interleukin 1 beta.

15. The method of claim 14, said method further comprising testing said cells having the hepatocyte phenotype for one or more of alkaline phosphatase, alpha fetoprotein, albumin, c-reactive protein, hepatocyte growth factor receptor, complement factor VII, complement factor IX, nestin, SOX-17, P450 CYP 1A2, P450 CYP 3A4, asialo-glycoprotein receptor 1, hepatocyte nuclear factor-4, GATA-4 and alpha-1-antitrypsin.

16. An article of manufacture comprising the composition of claim 1, wherein said composition is housed in a container.

17. The article of manufacture of claim 16, wherein said container is a vial, bottle, or a bag.

18. A kit comprising: a) the composition of claim 1, wherein said composition is housed in a container, and b) a clonal population of cells having the hepatocyte phenotype, the phenotype comprising alkaline phosphatase, alpha fetoprotein, albumin, c-reactive protein, hepatocyte growth factor receptor, complement factor VII, complement factor IX, nestin, SOX-17, P450 CYP 1A2, P450 CYP 3A4, asialo-glycoprotein receptor 1, hepatocyte nuclear factor-4, GATA-4, and alpha-1-antitrypsin.

19. The kit of claim 18, wherein said clonal population of cells is cryopreserved.

20. The kit of claim 19, wherein said cells are cryopreserved with fetal bovine serum, human serum, or human serum albumin in combination with one or more of the following: DMSO, trehalose, and dextran.
